(19) United States
(12) Patent Application Publication
(10) Pub. No.: US 2010/0105746 A1
(43) Pub. Date: Apr. 29, 2010

(54) METHOD FOR TREATING METABOLIC DISEASES

(76) Inventors: Clary Clish, Reading, MA (US); Shweta Verma, Hodson, MA (US); Sanjeev Forsyth, Milton, MA (US); Amanda Souza, Boston, MA (US); Nancy Finkel, Brighton, MA (US); Todor Dimitrov, Brookline, MA (US); David White, Norwell, MA (US)

Correspondence Address: HARNESS, DICKEY, & PIERCE, P.L.C, 7700 Bonhomme, Suite 400, ST. LOUIS, MO, 63105, US

(21) Appl. No.: 12/512,146

(22) Filed: Jul. 30, 2009

Related U.S. Application Data
(60) Provisional application No. 61/085,601, filed on Aug. 1, 2008.

(51) Int. Cl. A61K 31/421; A61P 3/00

(52) U.S. Cl. 514/374

(57) ABSTRACT

A method for treating a condition of low metabolic rate, dyslipidemia, excess adiposity and/or insulin resistance, or a disease or syndrome characterized by one or more of said conditions in a subject, comprises administering to the subject a therapeutically effective amount of romazarit or a pharmaceutically acceptable salt, ester or prodrug thereof.

This application claims the benefit of U.S. provisional application Ser. No. 61/085,601 filed on Aug. 1, 2008, the disclosure of which is incorporated by reference herein in its entirety.

### FIELD OF THE INVENTION

The present invention relates to methods for treating conditions of low metabolic rate, dyslipidemia, excess adiposity and/or insulin resistance, and diseases and syndromes characterized by one or more such conditions. More particularly, the invention relates to methods for treating dyslipidemia, obesity or a combination thereof as occurs, for example, in metabolic syndrome. More particularly, the invention relates to such methods comprising administering a pharmacotherapeutic agent.

### BACKGROUND

Dyslipidemia (a term used interchangeably with hyperlipidemia herein) is a metabolic condition in which plasma levels of lipids, primarily cholesterol and/or triglycerides, are elevated, and/or in which plasma levels of a particular fraction of cholesterol, namely HDL (high density lipoprotein) cholesterol, are depressed. Dyslipidemia, if untreated, typically leads to atherosclerosis, a hardening of arterial walls resulting from an inflammatory response to accumulation of lipid-containing plaque in the blood vessels. Atherosclerosis and plaque accumulation in turn are major risk factors for cardiovascular diseases such as heart attack, stroke, coronary artery disease and peripheral vascular disease.

Dyslipidemia can be primary or secondary. Primary causes of dyslipidemia include genetic disorders characterized by overproduction or defective clearance of triglycerides and/or LDL (low density lipoprotein) cholesterol, or in underproduction or excessive clearance of HDL. (LDL contributes to plaque and atherosclerosis; HDL is involved in removal of lipids and is protective against excessive plaque accumulation.) Primary lipid disorders are the most common cause of dyslipidemia in children, but do not cause a large percentage of cases in adults.

Most cases of dyslipidemia in adults are secondary. The most important secondary cause in developed countries is a sedentary lifestyle with excessive dietary intake of saturated fat, cholesterol and trans fatty acids (TFAs). Other common secondary causes include diabetes mellitus, alcohol overuse, chronic renal insufficiency and/or failure, hypothyroidism, primary biliary cirrhosis and other cholestatic liver diseases, and certain drugs including thiazides, beta-blockers, retinoids, highly active antiretroviral agents, estrogen and progestins, and glucocorticoids.

Diabetes is an especially significant secondary cause because patients tend to have a combination of high triglycerides, high LDL and low HDL. Patients with type 2 diabetes are especially at risk. Poor control of diabetes can lead to an increase in blood levels of free fatty acids, which in turn result in increased production of a triglyceride-rich lipoprotein fraction known as VLDL (very low density lipoprotein). Diabetic dyslipidemia is often exacerbated by increased caloric intake and physical inactivity, which characterize the lifestyles of some patients with type 2 diabetes. Women with diabetes are believed to be at special risk for cardiac disease from this form of dyslipidemia.

Dyslipidemia is diagnosed by measuring serum lipids. Routine measurements include total cholesterol, triglycerides, HDL and LDL, preferably in a fasting state. Total cholesterol, triglycerides and HDL are typically measured directly, and LDL levels are calculated from the other parameters by the Friedewald formula:

LDL=total cholesterol−(HDL+triglycerides/5).

LDL can also be measured directly using plasma ultracentrifugation and by an immunoassay method.

What constitutes a healthy lipid profile depends on other cardiovascular risk factors, but for most adults total cholesterol <200 mg/dl, triglycerides <150 mg/dl and HDL >50 mg/dl is generally desirable.

Another metabolic condition having serious implications for cardiovascular and other aspects of health is overweight or obesity, which can occur together with, or independently of, dyslipidemia. Definitions of overweight and obesity are most commonly based on body mass index (BMI), which is the ratio of body weight (expressed in kilograms) to the square of height (expressed in meters). As defined herein, and in accordance with the U.S. Centers for Disease Control and Prevention (CDC), an overweight adult has a BMI of 25-29.9, and an obese adult has a BMI≧30. A BMI≧40 is indicative of a condition sometimes called “morbid obesity” or “extreme obesity”. For children, the definitions of overweight and obese take into account age and gender effects on body fat. See, for example, CDC: Overweight and Obesity, www.cdc.gov/nccdphp/dnpa/obesity/defining.htm.

The percentage of the world's people that are overweight or obese is growing at an alarming rate. Obesity is already, or threatens to become, a major public health crisis in many countries, especially the developed countries of North America, Europe and Oceania. Rapid economic development in other parts of the world, including Asia and Latin America, is expected to lead to a great increase in prevalence of overweight and obesity in coming years. Already over 1.1 billion people worldwide are overweight or obese. In the United States alone, well over 50% of the adult population is considered overweight and about 30% is clinically obese. Obesity is also becoming a serious problem in American children. Overall, obesity is estimated to affect over 90 million people in the United States, a number that is predicted to continue to increase.

While overweight and obesity present problems in their own right (for example restriction of mobility, discomfort in tight spaces such as theater or airplane seats, social difficulties, etc.), their most serious consequences, particularly in the case of clinical obesity, arise from effects of obesity on other aspects of health. The estimated annual mortality from obesity-related diseases in the United States is over 300,000. See, for example, Hill & Peters (1998) Science 280:1371-1374.

Diseases and other adverse health conditions associated with or exacerbated or precipitated by obesity (referred to herein as “co-morbidities” of obesity) include cardiovascular disorders such as hypertension, dyslipidemia, ischemic heart disease, cardiomyopathy, cardiac infarction, stroke, venous thromboembolic disease and pulmonary hypertension; endocrine disorders such as type 2 diabetes; certain cancers including breast, prostate, bowel and endometrial cancers; respiratory disorders such as obesity-hypoventilation syndrome, asthma and obstructive sleep apnea; skeletal disorders such as low back pain and osteoarthritis of weight-bearing joints; psychiatric disorders such as depression; reproductive disorders such as sexual dysfunction (including erectile dysfunction), infertility, obstetric complications and fetal abnormalities; and many others, including gallstones, gout, non-alcoholic steatohepatitis (NASH), urinary incontinence, gastroesophageal reflux, venous and stasis ulcers, intracranial hypertension, accident proneness and skin disorders. See O'Brien & Dixon (2002) Amer. J. Surg. 184:4S-8S.

Obesity is fundamentally a disorder of energy balance. When food-derived energy (caloric intake) chronically exceeds energy expenditure, the excess energy is stored as triglycerides (fat) in adipose tissues, which readily expand to accommodate the added fat. Such expansion can involve increase in number (hyperplasia) or size (hypertrophy), or in both number and size, of individual cells (adipocytes) in adipose tissues. Several hormones and cytokines, including leptin and neuropeptide Y, are involved in controlling formation and development of adipose tissue. Mutations of such hormones or cytokines or their receptors can result in inactivation, leading to “genetic obesity” in humans and in animal models such as mice. Effects of disease on metabolism can also result in development of obesity. Commonly, however, obesity results from dietary excess in absence of controlling genetic or disease factors.

Factors contributing to development of obesity or metabolic syndrome thus include genetic factors, including ethnicity; factors related to age and sex, including hormonal changes that occur, for example, in pregnancy, at menopause or due to oral contraceptives or hormone replacement therapy; cultural and socioeconomic factors; psychological and behavioral factors including eating disorders; dietary habits, especially caloric intake and percentage of caloric intake from fats; tube-feeding; smoking cessation; physical activity, in particular energy expenditure (for example during physical work or exercise); disease factors; and side-effects of medication. Diseases and disorders tending to promote obesity illustratively include hypothyroidism (including Hashimoto's thyroiditis), Cushing syndrome, hypothalamic disorders, hypogonadism, pseudohypoparathyroidism, insulinoma, growth hormone deficiency, Prader-Willi and related genetic syndromes, polycystic ovarian syndrome, eating disorders such as binge-eating disorder, bulimia nervosa and night-eating disorder, and the like. Medications that can promote obesity in certain individuals include without limitation phenothiazines, valproate, carbamazepine, tricyclic antidepressants, lithium, glucocorticoids, megestrol acetate, thiazolidinediones, sulfonylureas, insulin, adrenergic antagonists, and serotonin antagonists such as cyproheptadine.

Excess adipogenesis can occur selectively in different parts of the body, and development of adipose tissue can be more dangerous to health in some parts than in others. Central obesity, typically associated with an “apple-shaped” body, results from excess adiposity especially in the abdominal region, including belly fat and visceral fat, and carries higher risk of co-morbidity than peripheral obesity, which is typically associated with a “pear-shaped” body resulting from excess adiposity especially on the hips. Measurement of waist/hip circumference ratio (WHR) can be used as an indicator of central obesity. A minimum WHR indicative of central obesity has been variously set, but a centrally obese adult as defined herein has a WHR≧0.85 if female and ≧0.9 if male.

Where adiposity is moderately in excess, as in overweight subjects, a regimen of diet and exercise to bring energy intake and expenditure into better balance is often enough to provide the desired weight loss. However, medication and/or other intervention can be helpful, together with an appropriate diet and exercise regimen, in achieving a target weight loss in an overweight subject. Furthermore, many overweight subjects may be physically unable, for example because of co-existing medical conditions or disabilities, to obtain sufficient exercise, and can benefit from anti-obesity medication.

When excess adiposity develops to a degree resulting in clinical obesity (BMI≧30), diet and exercise alone are usually not sufficient, thus for obese subjects medical and/or surgical intervention is often necessary, for example if serious risk from co-morbidities is to be avoided.

Co-occurrence of dyslipidemia and obesity can be more damaging to health and quality of life than either of these conditions alone. Sometimes dyslipidemia and obesity are accompanied by still other conditions that further exacerbate risk of cardiovascular and other diseases.

Metabolic syndrome, also known as syndrome X, insulin-resistance syndrome, Reaven's syndrome and CHAOS (an acronym for coronary artery disease, high blood pressure, adult onset (type 2) diabetes, obesity and stroke), is a combination of disorders affecting individuals having a diabetic or pre-diabetic condition such as impaired glucose tolerance or insulin resistance. A precise definition of metabolic syndrome is not generally agreed by all physicians; indeed it is not universally accepted as a true syndrome (as opposed to merely a collection of coexisting conditions). For purposes of the present disclosure, metabolic syndrome is defined as presence in a subject of at least one diabetic or prediabetic condition (for example type 2 diabetes, impaired glucose tolerance, elevated fasting plasma glucose—e.g., ≧100 mg/dl—or insulin resistance) together with at least two of the following:
elevated systolic and/or diastolic blood pressure, for example ≧130/80 mmHg, or on antihypertensive medication;
dyslipidemia involving one or more of
   elevated plasma triglycerides, for example ≧150 mg/dl, or
   depressed plasma HDL (high-density lipoprotein cholesterol), for example ≦50 mg/dl,
or on antihyperlipidemic medication;
central (also known as visceral or apple-shaped) obesity, for example BMI (body mass index) >30 kg/m² and/or WHR (waist to hip circumference ratio) >0.9 (male) or >0.85 (female); and
microalbuminuria, for example urinary albumin excretion ≧110 mg/dl or albumin/creatinine ratio ≧30 mg/g.

Additional signs occurring in some individuals with metabolic syndrome include one or more of elevated plasma uric acid, fatty liver, sometimes progressing to a condition known as NASH (non-alcoholic steatohepatitis), polycystic ovarian syndrome (in women), hemochromatosis (iron overload), and acanthosis nigricans, a skin condition featuring dark patches.

In a particular subpopulation of subjects with metabolic syndrome, biomarkers of systemic inflammation are elevated, indicating involvement of an inflammatory or pro-inflammatory process in the syndrome. Examples of such biomarkers include C-reactive protein (CRP), fibrinogen, interleukin-6 (IL-6) and tumor necrosis factor alpha (TNFα).

Individuals with metabolic syndrome are at elevated risk of developing full-blown type 2 diabetes and also of atherosclerotic vascular disease, including heart attack, stroke and kidney disease. These elevated risks in turn can result in reduced quality and length of life.

Metabolic syndrome can occur without overweight or obesity, but the obesity epidemic in “developed” countries has been paralleled by a similarly dramatic increase in incidence of metabolic syndrome. It is estimated that 22-25% of the population of North America has metabolic syndrome. A diagnosis of metabolic syndrome is being made in more and more younger people.

Among drugs that have shown promise for treatment of at least certain aspects of metabolic syndrome, especially the dyslipidemic component, are a class of lipid-modifying drugs known as fibrates. They include bezafibrate, fenofibrate and gemfibrozil, and are believed to act primarily via activation of peroxisome proliferator activated receptor alpha (PPARα). It has been suggested that fenofibrate, besides its antihyperlipidemic (especially triglyceride-lowering) effects, has other useful actions that make it a useful option for patients with dyslipidemias associated with diabetes, metabolic syndrome or HIV infection. See, for example, Tsimihodimos et al. (2005) Curr. Vase. Pharmacol. 3:87-98.

U.S. Patent Application Publication No. 2006/0083783 of Doyle et al. proposes a method for treating metabolic syndrome in a subject, comprising administering a therapeutically effective amount of fenofibrate to the subject.

Among compounds that have been disclosed to have antihyperlipidemic activity, and have been described as “fibrates”, is 2-[4-(4-chlorophenyl)benzyloxy]-2-methylpropanoic acid (clobuzarit). See, for example, Holloway & Thorp (1993) in Gibson & Lake, eds., Peroxisomes: Biology and Importance in Toxicology and Medicine, Chap. 19, pp. 449-464. London: Taylor & Francis.

Subsequent to a finding that clobuzarit, although originally developed as an antihyperlipidemic agent, had a spectrum of effects similar to that of disease-modifying antirheumatic drugs (DMARDs), Self et al. (1991) reported synthesis of a series of substituted heterocyclic alkoxypropionic acids and testing of these in animal models of chronic inflammation. One of the tested compounds was identified as 2-[[2-(4-chlorophenyl)-4-methyl-5-oxazolyl]methoxy]-2-methylpropanoic acid (romazarit; also known as Ro 31-3948).

Detailed studies of anti-inflammatory action of romazarit were reported by Bloxham et al. (1990) J. Pharmacol. Exp. Ther. 252:1331-1340, who were “particularly interested in compounds which were much weaker rodent peroxisomal proliferators” than clobuzarit, and stated that they selected romazarit on this basis. Efficacy in various models of chronic inflammation was reported. In its effect on hepatic PPA80, a specific marker of peroxisomal proliferation, romazarit was reportedly 16× less potent than clobuzarit.

### SUMMARY OF THE INVENTION

It has now been discovered that romazarit has unexpectedly strong activity in modulating a number of markers and physiological effects of low metabolic rate, in modulating blood lipids, in inhibiting weight gain, and in improving insulin sensitivity, all as described in greater detail hereinbelow. This unexpectedly strong and diverse activity, in combination with known anti-inflammatory activity, identifies romazarit for the first time as a drug with significant potential for treatment of conditions of low metabolic rate, dyslipidemia, excess adiposity and/or insulin resistance, and diseases and syndromes characterized by one or more such conditions.

Accordingly there is now provided a method for treating a condition of low metabolic rate, dyslipidemia, excess adiposity arid/or insulin resistance, or a disease or syndrome characterized by one or more such conditions, in a subject, the method comprising administering to the subject a therapeutically effective amount of romazarit or a pharmaceutically acceptable salt, ester or prodrug thereof.

Such a method is particularly useful wherein the condition, disease or syndrome is accompanied by systemic inflammation or presence of pro-inflammatory biomarkers.

In one embodiment the subject has dyslipidemia, obesity or a combination thereof.

In a particular embodiment, the subject is identified as having metabolic syndrome as defined herein.

### BRIEF DESCRIPTION OF THE DRAWINGS

FIG. 1 is a graph showing effects of romazarit at 5 and 50 mg/kg on plasma levels of different triglycerides in rats, as observed in a study described in Example 1 (initial experiment) herein. A key to the abbreviations used for different triglycerides is provided in Example 1.

FIG. 2 is a graph showing effects of romazarit at 15 and 50 mg/kg on plasma levels of different triglycerides in rats, as observed in a study described in Example 1 (second experiment) herein. A key to the abbreviations used for different triglycerides is provided in Example 1.

FIG. 3 is a graph showing effects of romazarit at 5 and 50 mg/kg on plasma levels of different cholesterol esters in rats, as observed in a study described in Example 1 (initial experiment) herein. A key to the abbreviations used for different cholesterol esters is provided in Example 1.

FIG. 4 is a graph showing effects of romazarit at 15 and 50 mg/kg on plasma levels of different cholesterol esters in rats, as observed in a study described in Example 1 (second experiment) herein. A key to the abbreviations used for different cholesterol esters is provided in Example 1.

FIG. 5 is a graph showing effects of romazarit at 5 and 50 mg/kg on plasma levels of free amino acids in rats, as observed in a study described in Example 2 (initial experiment) herein.

FIG. 6 is a graph showing effects of romazarit at 15 and 50 mg/kg on plasma levels of free amino acids in rats, as observed in a study described in Example 2 (second experiment) herein.

FIG. 7 is a graph showing effects of romazarit at 5 and 50 mg/kg on plasma levels of triiodothyronine (T₃) and thyroxine (T₄) in rats, as observed in a study described in Example 2 (initial experiment) herein.

FIG. 8 is a graph showing effects of romazarit at 15 and 50 mg/kg on plasma levels of triiodothyronine (T₃) and thyroxine (T₄) in rats, as observed in a study described in Example 2 (second experiment) herein.

FIG. 9 is a graph showing effects of romazarit at 5 and 50 mg/kg on weight gain in rats, as observed in a study described in Example 3 (initial experiment) herein.

FIG. 10 is a graph showing effects of romazarit at 15 and 50 mg/kg on weight gain or loss in rats, as observed in a study described in Example 3 (second experiment) herein.

FIG. 11 is a graph showing effects of romazarit, gemfibrozil and sibutramine on mean body weight of rats, as observed in a study described in Example 4 herein.

FIG. 12 is a graph showing effects of romazarit, gemfibrozil and sibutramine on food consumption by rats, as observed in a study described in Example 4 herein.

FIG. 13 is a graph showing effects of romazarit, gemfibrozil and sibutramine on plasma levels of glucose, total cholesterol and HDL cholesterol in rats, as observed in a study described in Example 5 herein.

FIG. 14 is a graph showing effects of romazarit, gemfibrozil and sibutramine on plasma levels of triglycerides in rats, as observed in a study described in Example 5 herein.

FIG. 15 is a graph showing effects of romazarit at 10, 50 and 100 mg/kg on plasma levels of different triglycerides in rats, as observed in a study described in Example 8 herein. A key to the abbreviations used for different triglycerides is provided in Example 8.

FIG. 16 is a graph showing effects of romazarit by comparison with bezafibrate, fenofibrate and gemfibrozil on plasma levels of different triglycerides in rats, as observed in a study described in Example 8 herein. A key to the abbreviations used for different triglycerides is provided in Example 8.

FIG. 17 is a graph showing effects of romazarit at 10, 50 and 100 mg/kg on plasma levels of different cholesterol esters in rats, as observed in a study described in Example 8 herein. A key to the abbreviations used for different cholesterol esters is provided in Example 8.

FIG. 18 is a graph showing effects of romazarit by comparison with bezafibrate, fenofibrate and gemfibrozil on plasma levels of different cholesterol esters in rats, as observed in a study described in Example 8 herein. A key to the abbreviations used for different cholesterol esters is provided in Example 8.

FIG. 19 is a graph showing effects of romazarit at 10, 50 and 100 mg/kg on plasma levels of free fatty acids in rats, as observed in a study described in Example 8 herein. EPA=eicosapentaenoic acid; DHA=docosahexaenoic acid.

FIG. 20 is a graph showing effects of romazarit by comparison with bezafibrate, fenofibrate and gemfibrozil on plasma levels of free fatty acids in rats, as observed in a study described in Example 8 herein. EPA=eicosapentaenoic acid; DHA=docosahexaenoic acid.

FIG. 21 is a graph showing effects of romazarit by comparison with bezafibrate, fenofibrate and gemfibrozil on liver weight in rats, as observed in a study described in Example 8 herein.

FIG. 22 is a graph showing effects of romazarit by comparison with bezafibrate, fenofibrate and gemfibrozil on plasma levels of adiponectin in rats, as observed in a study described in Example 9 herein.

FIG. 23 is a graph showing effects of romazarit by comparison with bezafibrate, fenofibrate and gemfibrozil on plasma levels of glucose in rats, as observed in a study described in Example 9 herein.

FIG. 24 is a graph showing effects of romazarit by comparison with bezafibrate, fenofibrate and gemfibrozil on plasma levels of insulin in rats, as observed in a study described in Example 9 herein.

FIG. 25 is a graph showing effects of romazarit by comparison with bezafibrate, fenofibrate and gemfibrozil on plasma levels of glucagon in rats, as observed in a study described in Example 9 herein.

FIG. 26 is a graph showing activation of PPARα (rat and human isoforms) by romazarit, as observed in a study described in Example 10 herein.

### DETAILED DESCRIPTION

A method is provided herein for treating a condition of low metabolic rate, dyslipidemia, excess adiposity and/or insulin resistance, or a disease or syndrome characterized by one or more such conditions in a subject. A “subject” herein can be of any animal species, more particularly any mammalian species including primates, farm and work animals such as horses, domestic pets such as dogs and cats, exotic animals including captive and zoo animals, laboratory animals such as rats, mice and other rodents, etc. Preferably the subject is a primate, more especially a human subject. Human subjects can be of either gender and of any age. A human subject who can benefit from practice of the present method is typically, but not necessarily, a patient under the care of a physician or clinician who can be a generalist or a specialist such as an endocrinologist. A patient can be in the community or in a residential care facility.

The phrase “low metabolic rate” herein means presence of one or more biomarkers or outward signs indicating that one or more metabolic, more especially catabolic, processes is reduced in rate, efficiency, capacity or response to stimuli by comparison with a normal healthy individual of the same gender and age group.

A “syndrome” herein refers to a complex of symptoms that occur together and that may be (but is not necessarily) reflective of a single underlying or causal disease or disorder. In some situations there may be mutual reinforcement of different symptoms. The terms “disorder” and “disease” are used interchangeably herein, unless the particular context demands that a distinction be drawn.

Unless the context demands otherwise, the terms “treat,” “treating” or “treatment” herein include preventive or prophylactic use of an agent in a subject at risk of, or having a prognosis including, a condition of low metabolic rate, dyslipidemia, excess adiposity and/or insulin resistance, or a disease or syndrome characterized by one or more such conditions, as well as use of such an agent in a subject already experiencing such a condition, disease or syndrome. Thus treatment includes (a) preventing development of such a condition, disease or syndrome from occurring in a subject that may be predisposed thereto but in whom the condition, disease or syndrome has not yet been diagnosed, or in a subject for whom development of such a condition, disease or syndrome would be highly hazardous due to presence of other complicating factors; (b) inhibiting progression of the condition, disease or syndrome or of one or more signs or symptoms thereof; (c) ameliorating or correcting an underlying dysfunction; and/or (d) ameliorating one or more symptoms without necessarily addressing an underlying dysfunction. The terms “prevent,” “preventing,” “prevention” and “preventive” will be understood to have their normal meaning in the medical arts of reducing risk or future incidence or severity of a disorder, or of one or more symptoms thereof, as opposed to total elimination of future occurrence of the disorder or symptoms.

Diseases or syndromes treatable by a method of the invention include those having one or more of the following conditions:
dyslipidemia, including elevated plasma triglycerides, elevated total cholesterol and/or depressed HDL cholesterol;
elevated plasma amino acids;
elevated plasma thyroxine and/or depressed plasma triiodothyronine;
elevated fasting glucose and/or impaired glucose tolerance (as indicated, for example, by high glucose excursion following glucose challenge);
insulin resistance or impaired insulin sensitivity; and/or
excess adiposity, especially central or visceral adiposity or outward signs thereof such as increased body weight, BMI and/or WHR.

As demonstrated in Example 10 hereinbelow, romazarit has now been found to have PPARα agonist activity. Without being bound by theory, it is believed that at least some of the effects of romazarit described herein are mediated, at least in part, by PPARα agonism. In one non-limiting embodiment, a condition, disease or syndrome treatable by a method of the invention has one or more conditions, for example one or more of the conditions listed immediately above, responsive to PPARα agonism.

Examples of conditions, diseases and syndromes treatable by a method of the invention include without limitation pre-diabetic conditions, obesity, dyslipidemia, NASH and metabolic syndrome. In one embodiment, the subject treated according to a method of the invention has dyslipidemia, obesity or a combination thereof.

The present method is contemplated to be especially useful in treatment of metabolic syndrome, in part because of (a) the growing prevalence of metabolic syndrome, (b) the distressing effects of metabolic syndrome on quality and length of life, including through its action as a predisposing factor to cardiovascular diseases such as heart attack and stroke, (c) the limited range of effective pharmacotherapies for metabolic syndrome, and (d) the surprising range of physiological effects exhibited by romazarit, which correspond remarkably closely to the range of symptoms typically seen in metabolic syndrome.

It will be recognized that, even though metabolic syndrome is not universally recognized as a true syndrome, the combination of conditions typically embraced by the term “metabolic syndrome” is sufficiently well described herein and in the literature to enable a subject who would benefit from treatment by a method of the present invention to be readily identified by the ordinarily skilled physician or clinician.

Conditions, diseases and syndromes having an inflammatory or pro-inflammatory component, a class that includes a high percentage of metabolic syndrome cases, are particularly amenable to treatment by the present method, in light of romazarit's known anti-inflammatory properties.

Romazarit can be administered to a subject in need thereof in the form of the free acid, or as a pharmaceutically acceptable salt, ester or prodrug thereof. Suitable salts include alkali metal or alkaline earth metal salts, for example sodium, potassium or magnesium salts; ammonium salt; and salts with organic airlines such as morpholine, thiomorpholine, piperidine, pyrrolidine, mono-, di- or tri-(C₁₋₆ alkylamines, for example ethylamine, tert-butylamine, diethylamine, diisopropylamine, triethylamine, tributylamine or dimethylpropylamine, or mono-, di- or tri-(hydroxy-(C_{1≢}alkyl)amines, for example monoethanolamine, diethanolamine or triethanolamine. The sodium salt (romazarit sodium) is an example of a particularly useful salt.

In some embodiments, a prodrug of romazarit or a salt of such prodrug can be used. A prodrug is a compound, typically itself having weak or no pharmaceutical activity, that is cleaved, metabolized or otherwise converted in the body of a subject to an active compound. Examples of prodrugs are esters, particularly alkyl esters and more particularly C₁₋₆ alkyl esters. Other examples include carbamates, carbonates, ketals, acetals, phosphates, phosphonates, sulfates and sulfonates.

Romazarit or a salt, ester or prodrug thereof is administered, according to the present invention, in a therapeutically effective amount. What constitutes a therapeutically effective amount can depend on a number of factors, including the particular disease or syndrome to be treated, the subject's age, gender and body weight, responsiveness of the particular subject and other factors, but will be readily established in individual situations by the ordinarily skilled physician or clinician without undue experimentation based on guidance provided herein. Doses herein are expressed in acid equivalent amounts of romazarit; where “romazarit” is mentioned herein it will be understood that equivalent amounts of a salt, ester or prodrug of romazarit can be substituted, unless the context demands otherwise.

In one embodiment, romazarit or a pharmaceutically acceptable salt, ester or prodrug thereof is administered in an amount effective to achieve at least one of (a) a reduction in plasma amino acids; (b) a reduction in plasma thyroxine and/or an increase in plasma triiodothyronine; (c) a reduction in plasma triglycerides; (d) an increase in plasma HDL or cholesterol esters indicative thereat (e) a reduction in fasting glucose level in plasma; (f) enhanced glucose tolerance; (g) an increase in plasma adiponectin (an indicator of insulin sensitivity); (h) reduction of excess visceral adiposity (as measured, for example, by WHR); and (i) inhibition of weight gain.

In various embodiments, the romazarit or salt, ester or prodrug thereof is administered in an amount effective to achieve at least two, or at least three, or at least four of the above effects.

The term “inhibition of weight gain” in the present context includes any of a reduction in the rate of body weight increase, stabilization of body weight (i.e., prevention of further increase), and body weight reduction (weight loss). Body weight can be expressed in absolute terms or as body mass index (BMI).

A daily (per diem) dose of romazarit useful herein can be titrated depending on the particular subject's response and on occurrence of any adverse side-effects. In most situations, a suitable daily dose is likely to be found in a range of about 1 to about 100 mg/kg body weight, for example about 2 to about 50 mg/kg body weight. For an adult human subject having a body weight of about 40 to about 100 kg, a suitable daily dose of romazarit can be, for example, about 50 to about 5000 mg, more typically about 100 to about 2500 mg or about 200 to about 1200 mg. Illustrative daily doses include, without limitation, about 100, about 150, about 200, about 250, about 300, about 400, about 500, about 600, about 750, about 1000, about 1200, about 1500, about 2000 or about 2500 mg.

The above doses are given on a per diem basis but should not be interpreted as necessarily being administered on a once daily frequency. Indeed the compound, or salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. The compound, or salt or prodrug thereof, can alternatively be administered more or less continuously, for example by parenteral infusion in a hospital setting. In some situations a single dose may be administered, but more typically administration is according to a regimen involving repeated dosage over a treatment period. In such a regimen the daily dose and/or frequency of administration can, if desired, be varied over the course of the treatment period, for example introducing the subject to the compound at a relatively low dose and then increasing the dose in one or more steps until a full dose is reached.

The treatment period is generally as long as is needed to achieve a desired outcome, for example a particular degree of improvement or attainment of a goal with respect to one or more parameters such as plasma chemistry parameters, body weight, BMI, etc. In some situations it will be found useful to administer romazarit intermittently, for example for treatment periods of days, weeks or months separated by non-treatment periods. In other situations it will be found useful to administer romazarit continuously and more or less indefinitely.

In practice of the present method, romazarit can be administered to the subject by any suitable route of administration. Routes of administration that efficiently deliver the romazarit to the circulatory system of the subject (“systemic routes”) are generally preferred. Systemic routes include without limitation parenteral, including intravenous (i.v.), subcutaneous (s.c.) and intradermal routes, transdermal routes, transmucosal, including rectal, intraoral and intranasal routes, and peroral (p.o.) routes. The term “oral” or “orally” applied to a route of administration herein will be understood to mean peroral, i.e., involving delivery to the gastrointestinal tract via the mouth, as opposed to intraoral, i.e., involving delivery across oral mucosa as in sublingual or buccal administration.

Romazarit is orally bioavailable, and oral administration is generally the most convenient route, especially for non-hospitalized patients.

While it can be possible to administer romazarit, whether as free base, salt, ester or prodrug, unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g., as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains romazarit in an amount of about 1% to about 99%, more typically about 5% to about 90% or about 10% to about 60%, by weight of the composition. A unit dosage form such as a tablet or capsule can conveniently contain an amount of romazarit providing a single dose, although where the dose required is large it may be necessary or desirable to administer a plurality of dosage forms as a single dose. For example, a unit dosage form can comprise romazarit in an amount of about 50 to about 2500 mg, for example about 100 to about 1500 mg or about 200 to about 1200 mg. Illustrative amounts of romazarit in a unit dosage form include, without limitation, about 100, about 150, about 200, about 250, about 300, about 400, about 500, about 600, about 750, about 1000, about 1200 or about 1500 mg.

The present invention has arisen in part from a number of insights and surprising findings that were not predictable from knowledge in the art relating to romazarit at the time the invention was made.

First, romazarit (I) has been compared most closely in the art with clobuzarit (II), to the extent of having been described as an “analog(ue)” thereof. See, for example, the article by Self et al. (1991) cited above.

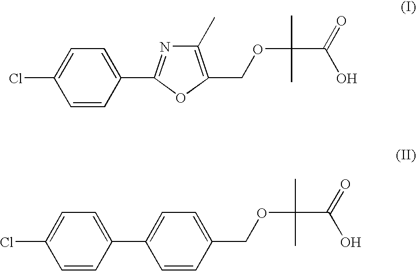

However, as indicated in the article by Bloxham et al. (1990) cited above, romazarit was selected for its good anti-inflammatory activity and its weak peroxisomal proliferative effect, about 16× weaker than clobuzarit in terms of induction of the PPA80 biomarker. Romazarit has therefore not heretofore been a promising candidate for use in treating metabolic conditions such as hyperlipidemia, for which agonists of the peroxisome proliferator activated receptor PPARα are known to be useful. Discovery by the present inventors that romazarit does exhibit PPARα agonist activity, and that it is an effective antihyperlipidemic in a rat model, permits a fundamental reappraisal of the therapeutic utility of this drug.

Second, as illustrated by data presented at least in Example 8 herein, the lipid modulating effects of romazarit are comparable, milligram for milligram, with those of well-known PPARα agonists such as gemfibrozil, fenofibrate and bezafibrate. Such substantial equipotency of romazarit with these established antihyperlipidemic drugs was not predictable, especially in view of romazarit's reportedly weak peroxisome proliferative potency (Bloxham et al. (1990), supra).

Third, even if the disclosure by Bloxham et al. (1990), supra of the weak peroxisome proliferative effect of romazarit were ignored and the compound were predicted, based on superficial similarities in chemical structure to fibrate drugs, to have PPARα agonist activity, it could not have been predicted that romazarit would exhibit such a spectrum of effects as demonstrated herein, including not only antihyperlipidemic effects but also effects on plasma amino acids, plasma thyroxine, fasting glucose, response to glucose challenge, indicators of insulin sensitivity, and weight gain.

For example, as shown in Examples 3 and 4 herein, romazarit has been found to reduce weight gain or, in rats with diet-induced obesity, provide weight loss. In a comparison with the PPARα agonist gemfibrozil (Example 4), romazarit at 50 mg/kg caused substantial weight reduction (at least comparable to the anti-obesity standard sibutramine) whereas gemfibrozil at 50 mg/kg gave no such effect. The anti-obesity effect of romazarit may be associated, at least in part, with a transient depression of food consumption, as shown in Example 4.

As another example of the unpredicted spectrum of effects, romazarit has been found to improve oral glucose tolerance in a dose-dependent fashion, in a test (OGTT) where no such improvement was seen with gemfibrozil. See Example 5 herein, the evidence of which indicates a potential utility for romazarit as an agent for enhancing glucose tolerance, a key diagnostic criterion of diabetes.

As yet another example of the unpredicted spectrum of effects, romazarit has been found to increase plasma adiponectin in a dose-dependent fashion, to a degree not seen with gemfibrozil, fenofibrate or bezafibrate. See Example 9 herein. Adiponectin is an insulin-sensitizing protein that is abundantly expressed in adipocytes and is secreted into the circulation (see Lara-Castro et al. (2007) Curr. Opin. Lipidol. 18:263-270). The evidence of Example 9 therefore indicates a potential utility for romazarit as an agent for enhancing insulin sensitivity.

Without being bound by theory, it is believed that romazarit is not a PPARγ activator like insulin-sensitivity-enhancing drugs of the thiazolidinedione class, and that its effect on adiponectin is mediated by a different mechanism. This is potentially important, noting that PPARγ agonists and dual PPARα/PPARγ agonists tend to be associated with weight gain (see Larsen et al. (2003) Diabetes 52:2249-2259).

Fourth, the surprisingly broad spectrum of effects of romazarit mentioned above, including antihyperlipidemic effect, anti-obesity effect, enhancement of glucose tolerance and enhancement of insulin sensitivity, mark this compound as having a remarkably good fit as a therapeutic agent for metabolic syndrome. Because of drug-drug interactions it is often preferable to minimize the number of different drugs administered to a patient, thus a single drug that simultaneously treats a variety of aspects of a syndrome such as metabolic syndrome offers a very important benefit. This is especially true where the spectrum of effects and the spectrum of disease conditions are perfectly matched and the romazarit can be administered in monotherapy. However, it remains true even if romazarit is used as part of a combination therapy, for the multifaceted or pleiotropic activity of romazarit can permit, at least in some situations, a reduction in dose of concomitantly administered drugs. In subjects having hypertension as a component of metabolic syndrome, it will generally be desirable to administer antihypertensive medication (one or more drugs such as diuretics, angiotensin-converting enzyme (ACE) inhibitors, angiotensin II receptor antagonists, β-adrenergic receptor antagonists, calcium channel blockers, etc.) concomitantly with romazarit.

And fifth, the combination of effects shown herein with the known effectiveness of romazarit in treating chronic systemic inflammation provides a still further benefit for subjects with dyslipidemia, obesity or a combination thereof as in metabolic syndrome, it being known that inflammatory processes are involved in at least a substantial percentage of such conditions.

As mentioned above, romazarit can be one of a plurality of active agents administered for treatment of a metabolic condition, disease or syndrome herein. In some cases, romazarit can be administered for treatment of one or more components of a disease or syndrome concomitantly with one or more additional active agents for treatment of other components or for treatment of an associated condition. In other cases, romazarit can be administered to mitigate a metabolic or obesity-promoting side-effect of other medications, for example phenothiazines, valproate, carbamazepine, tricyclic antidepressants, lithium, glucocorticoids, megestrol acetate, thiazolidinediones, sulfonylureas, insulin, adrenergic antagonists, and serotonin antagonists such as cyproheptadine. Each of these situations is referred to herein as combination therapy.

An “associated condition” herein can be one that is secondary to the disease or syndrome in question, for example hypertension, ischemic heart disease, cardiomyopathy, cardiac infarction, stroke, venous thromboembolic disease, pulmonary hypertension, certain cancers including breast, prostate, bowel and endometrial cancers, obesity-hypoventilation syndrome, asthma, obstructive sleep apnea, low back pain, osteoarthritis of weight-bearing joints, depression, sexual dysfunction (including erectile dysfunction), infertility, obstetric complications, fetal abnormalities, gallstones, gout, NASH, urinary incontinence, gastroesophageal reflux, venous and stasis ulcers, intracranial hypertension, accident proneness and skin disorders. Combination therapy with romazarit and one or more agents effective for treating any of the above secondary conditions is an embodiment of the present invention.

A still further embodiment of the present invention is a method for preventing (i.e., reducing risk or future incidence or severity of) any of the above associated conditions in a subject having dyslipidemia, obesity or a combination thereof (including metabolic syndrome), comprising administering to the subject romazarit or a pharmaceutically acceptable salt, ester or prodrug thereof in an amount therapeutically effective for treatment of the dyslipidemia, obesity or combination thereof, optionally in combination with one or more additional active agents as described herein.

Alternatively or in addition, an “associated condition” herein can be one to which the metabolic condition, disease or syndrome is secondary, for example a diabetic or pre-diabetic condition such as type 2 diabetes, hormonal changes resulting from pregnancy, menopause, oral contraceptives or hormone replacement therapy, eating disorders such as binge-eating disorder, bulimia nervosa and night-eating disorder, tube-feeding, smoking cessation, impaired physical activity, hypothyroidism (including Hashimoto's thyroiditis), Cushing syndrome, hypothalamic disorders, hypogonadism, pseudohypoparathyroidism, insulinoma, growth hormone deficiency, Prader-Willi and related genetic syndromes, or polycystic ovarian syndrome. Combination therapy with romazarit and one or more agents effective for treating any of the above conditions to which the romazarit-treatable condition, disease or syndrome is secondary is an embodiment of the present invention.

The two or more active agents administered in combination can be formulated in one pharmaceutical preparation (single dosage form) for administration to the subject at the same time, or in two or more distinct preparations (separate dosage forms) for administration to the subject at the same or different times, e.g., sequentially. The two distinct preparations can be formulated for administration by the same route or by different routes.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in a first container, a first agent that comprises romazarit or a pharmaceutically acceptable salt, ester or prodrug thereof, and, in a second container, a second agent as indicated above. In another example, the first and second agents are separately packaged and available for sale independently of one another, but are co-marketed or co-promoted for use according to the invention. The separate dosage forms may also be presented to a subject separately and independently, for use according to the invention.

Depending on the dosage forms, which may be identical or different, e.g., fast release dosage forms, controlled release dosage forms or depot forms, the first and second agents may be administered on the same or on different schedules, for example on a daily, weekly or monthly basis.

A therapeutic combination comprising romazarit or a pharmaceutically acceptable salt, ester or prodrug thereof, and an antidiabetic agent is a particular embodiment of the present invention. A method for treating metabolic syndrome in a subject, comprising administering such a therapeutic combination to the subject, is also a particular embodiment of the invention. The antidiabetic agent preferably has a mode of action that is different from or complementary to that of romazarit. The combination can comprise separate dosage forms of the romazarit and the antidiabetic agent, for example separately packaged or co-packaged, or can have both the romazarit and the antidiabetic agent co-formulated in the same dosage form. One or more antidiabetic agents can be present in the combination.

Suitable antidiabetic agents for such a combination include without limitation sulfonylureas, biguanides, thiazolidinediones, α-glucosidase inhibitors, incretin mimetics and hormones and analogs thereof. Illustrative antidiabetic agents include without limitation acarbose, acetohexamide, amylin, buformin, carbutamide, chlorpropamide, exenatide, glibornuride, glicazide, glimepiride, glipizide, gliquidone, glisoxepid, glyburide, glybuthiazole, glymidine, insulin, liraglutide, metformin, miglitol, mitiglinide, muraglitazar, nateglinide, phenformin, pioglitazone, pramlintide, repaglinide, rosiglitazone, sitagliptin, tesaglitzar, tolazamide, tolbutamide, tolcyclamide, troglitazone, vildagliptin and voglibose.

A therapeutic combination comprising romazarit or a pharmaceutically acceptable salt, ester or prodrug thereof, and an antihypertensive agent is a particular embodiment of the present invention. A method for treating metabolic syndrome in a subject, comprising administering such a therapeutic combination to the subject, is also a particular embodiment of the invention. The combination can comprise separate dosage forms of the romazarit and the antihypertensive agent, for example separately packaged or co-packaged, or can have both the romazarit and the antihypertensive agent co-formulated in the same dosage form. One or more antihypertensive agents can be present in the combination.

Suitable antihypertensive agents for such a combination include without limitation diuretics, ACE inhibitors, angiotensin II receptor antagonists, β-adrenergic receptor antagonists (beta-blockers), calcium channel blockers and vasodilators.

One or more diuretics can illustratively be selected from the following list:

### Organomercurials

chlormerodrin

chlorothiazide

chlorthalidone

meralluride

mercaptomerin sodium

mercumatilin sodium

mercurous chloride

mersalyl

### Purines

pamabrom

protheobromine

theobromine

### Steroids

canrenone

eplerenone

oleandrin

spironolactone

### Sulfonamide Derivatives

acetazolamide

ambuside

azosemide

bumetanide

butazolamide

chloraminophenamide

clofenamide

clopamide

clorexolone

disulfamide

ethoxzolamide

furosemide

mefruside

methazolamide

piretanide

torsemide

tripamide

xipamide

### Thiazides and Analogs

althiazide

bendroflumethiazide

benzthiazide

benzylhydrochlorothiazide

buthiazide

chlorthalidone

cyclopenthiazide

cyclothiazide

ethiazide

fenquizone

hydrochlorothiazide

hydroflumethiazide

indapamide

methyclothiazide

metolazone

paraflutizide

polythiazide

quinethazone

teclothiazide

trichlormethiazide

### Uracils

aminometradine

### Unclassified

amiloride

Biogen BG 9719

chlorazanil

ethacrynic acid

etozolin

isosorbide

Kiowa Hakko KW 3902

mannitol

muzolimine

perhexiline

Sanofi-Aventis SR 121463

ticrynafen

triamterene

urea

It is noted that thiazide diuretics are generally not preferred in patients with diabetes.

One or more ACE inhibitors can illustratively be selected from the following list:

alacepril

benazepril

captopril

ceronapril

cilazapril

delapril

enalapril

enalaprilat

eosinopril

fosinopril

imidapril

lisinopril

moexipril

moveltipril

omapatrilat

perindopril

quinapril

ramipril

sampatrilat

spirapril

temocapril

trandolapril

One or more angiotensin II receptor antagonists can illustratively be selected from the following list:

candesartan

eprosartan

irbesartan

losartan

olmesartan

tasosartan

telmisartan

valsartan

One or more beta-blockers can illustratively be selected from the following list:

AC 623

acebutolol

alprenolol

atenolol

amosulalol

arotinolol

atenolol

befunolol

betaxolol

bevantolol

bisoprolol

bopindolol

bucindolol

bucumolol

bufetolol

bufuralol

bunitrolol

bupranolol

butidrine hydrochloride

butofilolol

carazolol

carteolol

carvedilol

celiprolol

cetamolol

cloranolol

dilevalol

esmolol

indenolol

labetalol

landiolol

levobunolol

mepindolol

metipranolol

metoprolol

moprolol

nadolol

nadoxolol

nebivolol

nifenalol

nipradilol

oxprenolol

penbutolol

pindolol

practolol

pronethalol

propranolol

sotalol

sulfinalol

talinolol

tertatolol

tilisolol

timolol

toliprolol

xibenolol

One or more calcium channel blockers can illustratively be selected from the following list:

### Arylalkylamines

bepridil

clentiazem

diltiazem

fendiline

gallopamil

mibefradil

prenylamine

semotiadil

terodiline

verapamil

### Dihydropyridine Derivatives

amlodipine

aranidipine

barnidipine

benidipine

cilnidipine

efonidipine

elgodipine

felodipine

isradipine

lacidipine

lercanidipine

manidipine

nicardipine

nifedipine

nilvadipine

nimodipine

nisoldipine

nitrendipine

NZ 105

### Piperazine Derivatives

cinnarizine

dotarizine

flunarizine

lidoflazine

lomerizine

### Unclassified

bencyclane

etafenone

fantofarone

monatepil

perhexiline

One or more vasodilators can illustratively be selected from the following list:

amotriphene

benfurodil hemisuccinate

benziodarone

chloracizine

chromonar

clobenfurol

clonitrate

cloricromen

dilazep

droprenilamine

efloxate

erythrityl tetranitrate

etafenone

fendiline

hexestrol bis(β-diethylaminoethyl ether)

hexobendine

hydralazine

isosorbide dinitrate

isosorbide mononitrate

itramin tosylate

khellin

lidoflazine

mannitol hexanitrate

minoxidil

nitroglycerin

pentaerythritol tetranitrate

pentrinitrol

perhexiline

pimefylline

prenylamine

propatyl nitrate

trapidil

tricromyl

trimetazidine

trolnitrate phosphate

visnadine

Other antihypertensive agents that can optionally be used in combination with romazarit in certain circumstances include alpha-I -adrenergic receptor blockers, aldosterone receptor antagonists, endothelin receptor antagonists, vasopeptidase inhibitors, NEP (neutral endopeptidase) inhibitors and prostanoids.

One or more alpha-1-adrenergic receptor blockers can illustratively be selected from the following list:

amosulalol

arotinolol

carvedilol

dapiprazole

doxazosin

ergoloid mesylates

fenspiride

idazoxan

indoramin

labetalol

methyldopa

monatepil

naftopidil

nicergoline

prazosin

tamsulosin

terazosin

tolazoline

trimazosin

yohimbine

One or more aldosterone receptor antagonists can illustratively be selected from the following list:

canrenone

eplerenone

spironolactone

One or more endothelin receptor antagonists can illustratively be selected from the following list:

ambrisentan

atrasentan

avosentan

bosentan

clazosentan

darusentan

sitaxsentan

TBC-3711

tezosentan

One or more vasopeptidase inhibitors can illustratively be selected from the following list:

fasidotril

omapatrilat

sampatrilat

One or more NEP inhibitors, some of which are also ACE inhibitors, can illustratively be selected from the following list:

candoxatril

CGS 26582

MDL 100173

omapatrilat

phosphoramidon

sinorphan

thiorphan

Z13752A

One or more prostanoids can illustratively be selected from the following list:

beraprost

cicaprost

epoprostenol

iloprost

PGE₁

PGI₂ (prostacyclin)

NS-304

treprostinil

A therapeutic combination comprising romazarit or a pharmaceutically acceptable salt, ester or prodrug thereof, and an anti-obesity agent is a particular embodiment of the present invention. A method for treating a syndrome that includes dyslipidemia and obesity, for example metabolic syndrome, in a subject, comprising administering such a therapeutic combination to the subject, is also a particular embodiment of the invention. The anti-obesity agent preferably has a mode of action that is different from or complementary to that of romazarit. The combination can comprise separate dosage forms of the romazarit and the anti-obesity agent, for example separately packaged or co-packaged, or can have both the romazarit and the anti-obesity agent co-formulated in the same dosage form. One or more anti-obesity agents can be present in the combination.

Illustrative anti-obesity agents include without limitation aminorex, amphetamine, benzphetamine, chlorphentermine, clobenzorex, clortermine, cyclexedrine, dextroamphetamine, diethylpropion, N-ethylamphetamine, fenbutrazate, fenfluramine, fenproporex, levophacetoperane, mazindol, mefenorex, methamphetamine, norpseudoephedrine, orlistat, pentorex, phendimetrazine, phenmetrazine, phentermine, phenylpropanolamine, rimonabant and sibutramine.

A therapeutic combination comprising romazarit or a pharmaceutically acceptable salt, ester or prodrug thereof, and an antihyperlipidemic agent is a particular embodiment of the present invention. A method for treating a syndrome that includes dyslipidemia and obesity, for example metabolic syndrome, in a subject, comprising administering such a therapeutic combination to the subject, is also a particular embodiment of the invention. The antihyperlipidemic agent preferably has a mode of action that is different from or complementary to that of romazarit, in particular it is preferably other than a fibrate. The combination can comprise separate dosage foul's of the romazarit and the antihyperlipidemic agent, for example separately packaged or co-packaged, or can have both the romazarit and the antihyperlipidemic agent co-formulated in the same dosage form. One or more antihyperlipidemic agents can be present in the combination.

Suitable antihyperlipidemic agents for such a combination include without limitation bile acid sequestrants, HMG CoA reductase inhibitors (statins), niacin derivatives and cholesterol absorption inhibitors. Illustrative antihyperlipidemic agents other than fibrates include without limitation acifran, acipimox, aluminum nicotinate, atorvastatin, avasimibe, benfluorex, cerivastatin, cholestyramine resin, colesevalam, colestilan, colestipol, detaxtran, eicosapentaenoic acid, ezetimibe, fluvastatin, lovastatin, meglutol, melinamide, niacin, niceritrol, γ-oryzanol, oxiniacic acid, pantethine, pirozadil, pitavastatin, policozanol, polidexide, pravastatin, probucol, rosuvastatin, simvastatin, β-sitosterol, sultosilic acid, tiadenol, torcetrapib and xenbucin.

### Examples

The following working examples are illustrative of the present invention, and are not to be construed as limiting in any way.

### Example 1

### Romazarit Modulated Plasma Lipids in Rats

Multiplex bioanalytics profiling can elucidate novel pharmacological actions of drugs via measurement of hundreds of disease-relevant biomolecules in biofluids collected from drug-dosed animals. Romazarit was evaluated via a full multiplex bioanalytics profiling study.

Male Sprague-Dawley rats (CD IGS Charles River Laboratories; average weight 285 g at beginning of dosing) received romazarit suspended in a 0.5% carboxymethylcellulose (CMC)+0.2% polysorbate 80 (Tween™ 80) vehicle via oral gavage (p.o.) twice per day (b.i.d.) over a period of seven days. In an initial experiment, two dose groups of six animals per group received either 5 or 50 mg/kg b.i.d., and a further group received vehicle only, as a control. A follow-up experiment was performed to explore dose-response and the selected doses were 15 and 50 mg/kg b.i.d., again plus control.

In both experiments, urine was collected from each animal by clean-catch after the final dose on day 7, and cerebrospinal fluid (CSF) and plasma were collected (terminal samples) on day 8. Aliquots of the CSF and plasma samples from each individual were submitted for LC-MS analyses of bioanalytes. Three different separation methods were run, with MS analyses in both positive and negative ion modes, to enable resolution of relative quantification of the bioanalytes. These methods included:
RP-I: LC-MS of polar and non-polar abundant lipids (e.g., individual cholesterol esters, triglycerides, diacylglycerols, phospholipids, lysophospholipids);
RP-II: LC-MS of lower abundance bioanalytes (e.g., eicosanoids & other lipid mediators, fatty acids, oxidized fatty acids, bile acids, conjugated bile acids);
PLR: LC-MS of polar compounds (e.g., catecholamines & serotonin pathway, amino acids, organic acids, sugars).

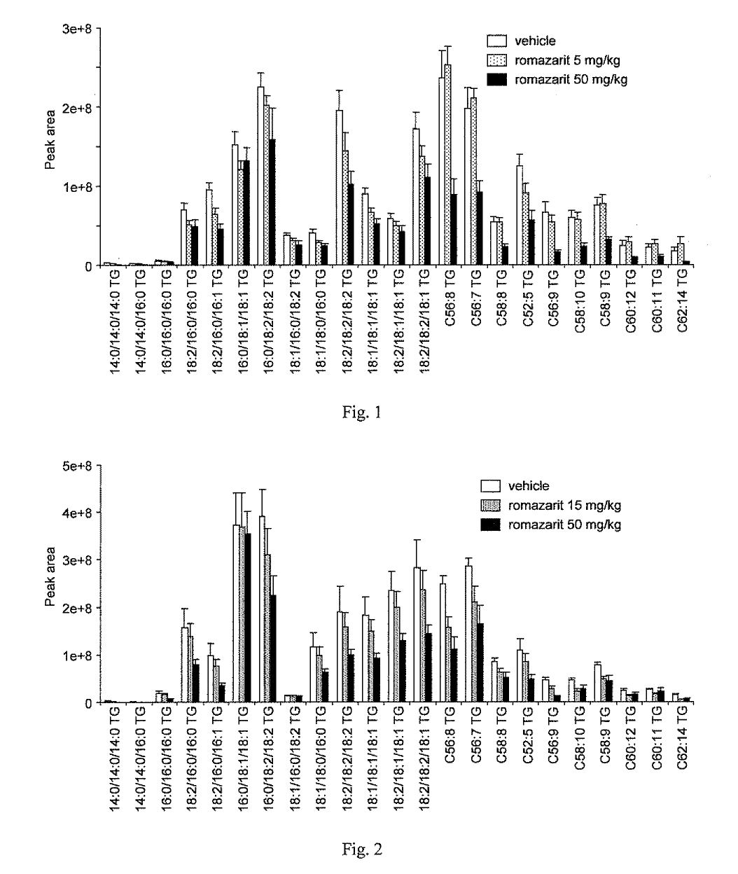

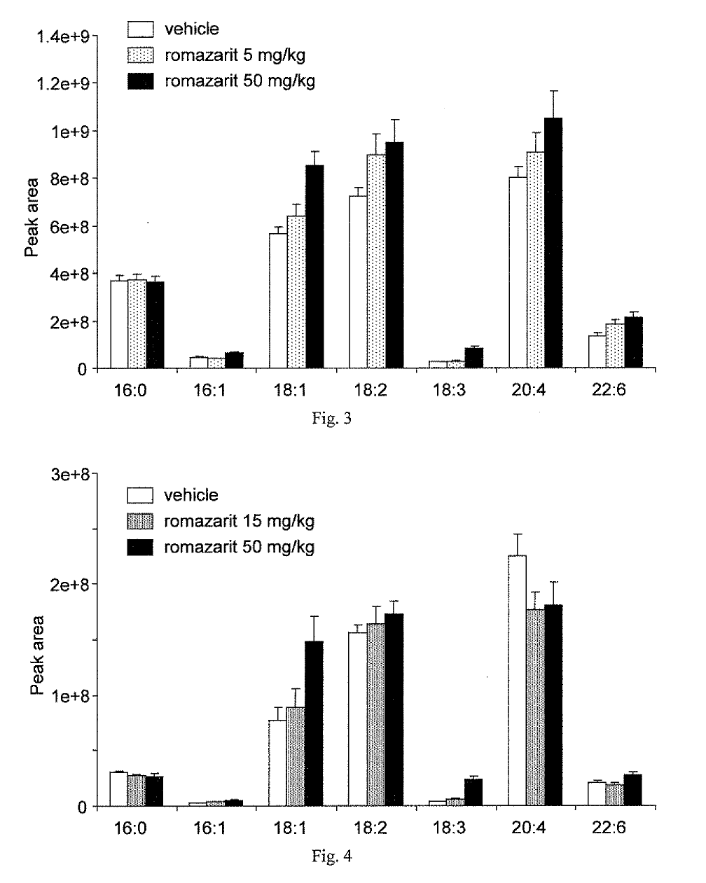

In the initial experiment, in which romazarit was administered at 5 and 50 mg/kg b.i.d., a dramatic alteration in the profile of plasma lipids was observed. For example, romazarit lowered plasma triglycerides (FIG. 1). This effect was highly pronounced in the 50 mg/kg group but was also apparent in the 5 mg/kg group. Plasma cholesterol esters, the levels of which correlate with and are an indicator of plasma HDL cholesterol in this model, were also increased by romazarit (FIG. 3).

In FIGS. 1 and 2, different triglyceride moieties are identified by abbreviations derived as follows. Fatty acyl carbon content and unsaturation are denoted as x:y where x=number of carbon atoms and y=number of double bonds. Where these parameters have been identified for each of the three constituent fatty acids in the triglyceride, they are shown individually. Where data are available only for the total number of acyl carbons and double bonds in all three constituent fatty acids, only one x:y descriptor is shown, representing the total in each case. In FIGS. 3 and 4, different cholesterol esters are identified by abbreviations derived as follows. Fatty acyl carbon content and unsaturation are denoted as x:y where x=number of carbon atoms and y=number of double bonds.

As the most dramatic effects of romazarit on metabolic parameters were observed in the 50 mg/kg b.i.d. dose group, the experiment was repeated with a substitution of 15 mg/kg b.i.d. for the 5 mg/kg b.i.d. dose. The 15 mg/kg dose lowered plasma triglycerides to a degree that was intermediate between the effects seen in the initial experiment of 5 mg/kg and 50 mg/kg doses, and the results for the 50 mg/kg group were generally similar to those seen with the same dose in the initial experiment (FIG. 2). The effects of the 15 mg/kg dose on cholesterol esters (FIG. 4) were similar to those of the 5 mg/kg dose utilized in the initial experiment, and again the effects of the 50 mg/kg dose were generally similar to those seen with the same dose in the initial experiment.

The results of this study provided for the first time an indication that romazarit, originally developed as an anti-rheumatic agent, has potential as an antidyslipidemic agent.

### Example 2

### Romazarit Lowered Plasma Amino Acids and Thyroxine in Rats

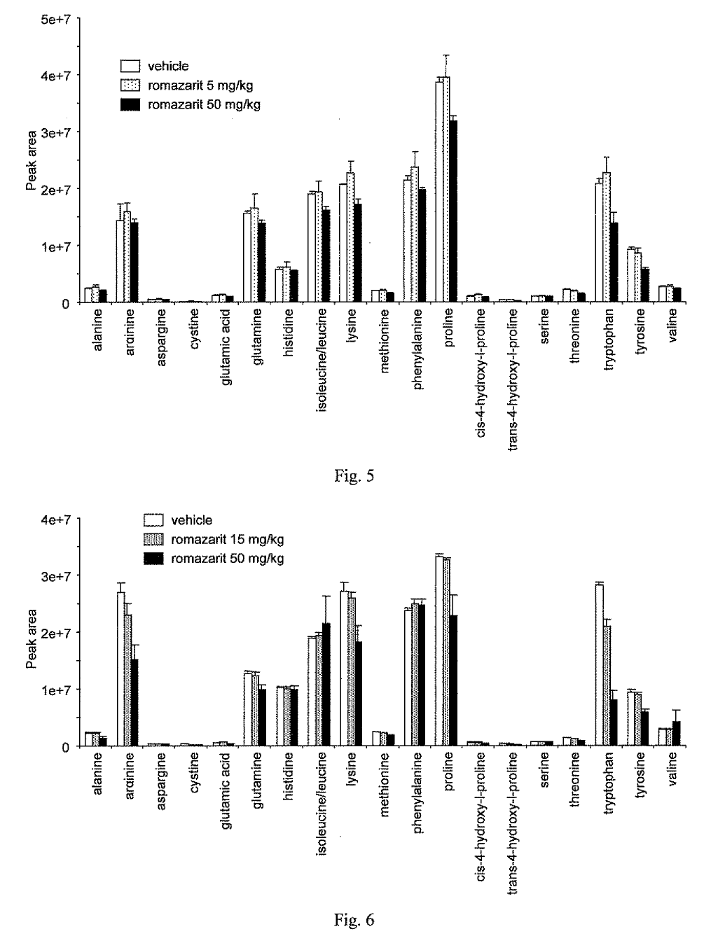

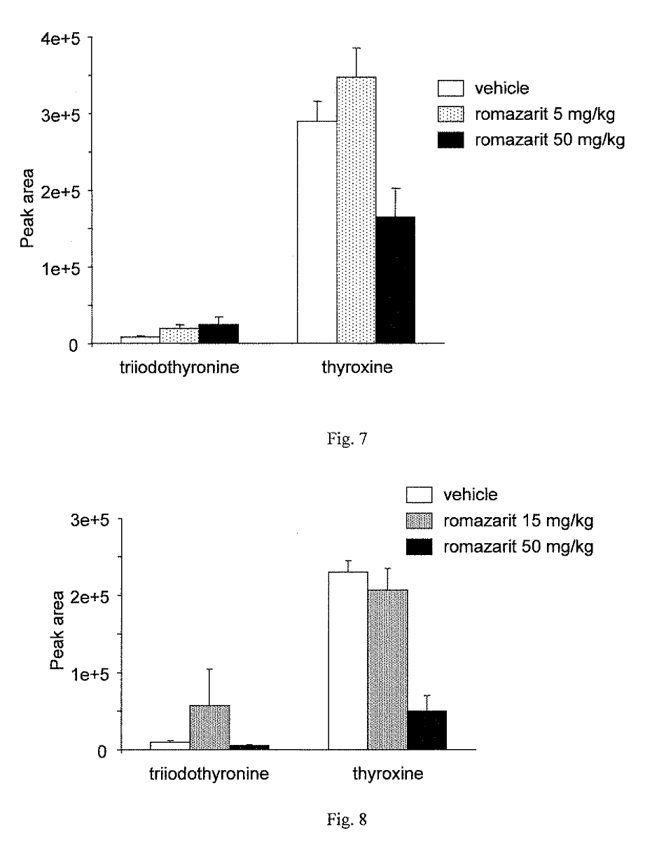

In the experiments described in Example 1, LC-MS analyses of polar bioanalytes revealed a small but significant lowering of levels of common amino acids in plasma (FIG. 5) in rats treated with romazarit. Lowered amino acids in plasma can be indicative of hypercatabolism and increased metabolic rate. A concomitant decrease in plasma thyroxine (T₄) was also observed (FIG. 7), lending further evidence that romazarit influences metabolic rate. In the second experiment, results for the 50 mg/kg group were generally similar to those for the same dose in the initial experiment for both plasma amino acids (FIG. 6) and plasma thyroxine (FIG. 8). The effects of the 15 mg/kg dose on plasma amino acids and plasma thyroxine were similar to those seen with the 5 mg/kg dose in the initial experiment.

The results of this study provided an indication that romazarit not only has antidyslipidemic effects as shown in Example 1, but has potential to increase metabolic rate in subjects having metabolic insufficiency as indicated by excessive plasma levels of amino acids and/or thyroxine.

### Example 3

### Romazarit Inhibited Weight Gain in Rats

In the experiments described in Example 1, romazarit was also evaluated for effects on weight gain in rats. Body weights of individual rats were recorded before and after the seven-day dosing period.

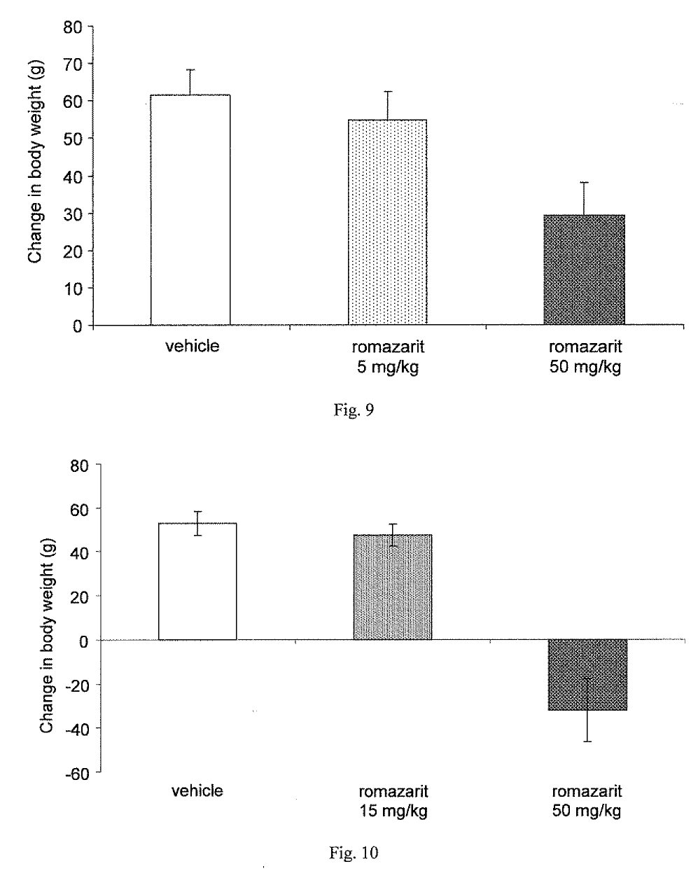

In the initial experiment, a dramatic inhibition of weight gain was observed in the 50 mg/kg romazarit dose group and a similar but weaker trend toward inhibition of weight gain was apparent in the 5 mg/kg group (FIG. 9). In the second experiment, inhibition of weight gain in the 15 mg/kg group was generally similar to that seen in the initial experiment with the 5 mg/kg dose. In this experiment, the 50 mg/kg dose not only inhibited weight gain, but even resulted in weight loss (FIG. 10).

The results of this study provided an indication that, in addition to the effects on plasma lipids, amino acids and thyroxine seen in Examples 1 and 2, romazarit, especially at higher doses, can inhibit weight gain or even provide weight loss in rats.

### Example 4

### Romazarit Provided Weight Loss in a Rat Model of Diet-Induced Obesity

### Objective of Study

The objective of this study was evaluation of dose-dependent efficacy of romazarit as measured by improvement in body weight and metabolic state in a diet-induced obese (DIO) rat model. Two positive controls were included as comparators, the lipid-lowering fibrate gemfibrozil and the centrally-acting neurotransmitter reuptake inhibitor sibutramine, available clinically as an anti-obesity medication.

### Study Methods

Male Sprague-Dawley rats were acclimated for 7 weeks on a high-fat diet before commencement of dose administration, and maintained on the same diet throughout the subsequent dosing period of 23 days. The high-fat diet (D12492 commercially available from Research Diets, Inc., New Brunswick, N.J.) was one in which at least 60% of calories are derived from fat.

Romazarit was administered at three dose levels: 15, 50 and 100 mg/kg. Dose levels of gemfibrozil (50 mg/kg) and sibutramine (10 mg/kg) were selected based on published reports. All treatments were administered once daily by oral gavage in 10 ml vehicle. A vehicle-only control was also administered. Each treatment group consisted of 10 animals.

Body weight of each animal was measured weekly during the 7-week acclimation period and five times weekly during the 23-day dosing period. Food consumption was recorded twice weekly during the 7-week acclimation period and five times weekly during the 23-day dosing period.

Animals were sacrificed and liver weights measured at conclusion of the study. It is believed that increased liver weight is an indicator of peroxisome proliferation in the liver, a known effect of PPARα agonists in rodents.

### Results

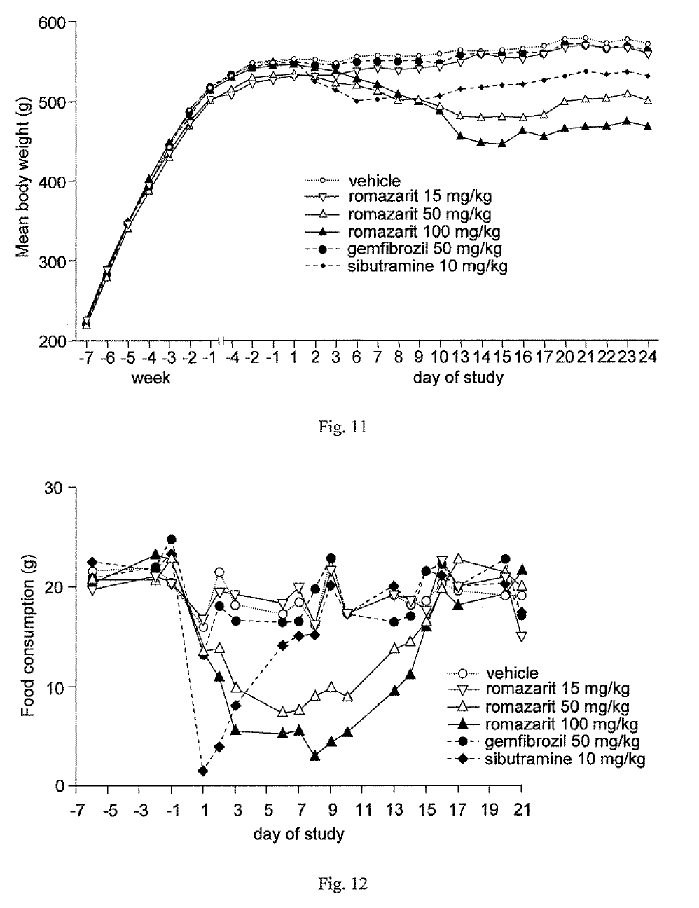

Changes in body weight are shown in FIG. 11. By the time the dosing regimen was initiated (day 1) the rats had attained close to their maximum weight. In this study, a dose-dependent effect of romazarit on body weight was apparent within the first week. At the highest dose level, 100 mg/kg, body weight loss by comparison with the vehicle control was significant, with a maximum weight loss of 16% seen on day 16, leveling off to 13.3% at the end of the study (day 23). At the 50 mg/kg dose level, a more moderate effect was seen, 10.3% at day 16, leveling off to 4.6% at day 23. At the lowest dose level, 15 mg/kg, mean body weight was similar to the vehicle control for the duration of the study. A similar, significant weight loss was observed with sibutramine, although the kinetics of weight loss and subsequent recovery were more rapid than with 50 or 100 mg/kg romazarit. Treatment with gemfibrozil had no significant effect on body weight throughout the dosing period.

Changes in food consumption are shown in FIG. 12. The romazarit treatment groups demonstrated a transient dose-dependent decrease in food consumption. At the lowest dose (15 mg/kg), food consumption was comparable to the vehicle control throughout the dosing period. Food consumption by animals receiving 50 and 100 mg/kg romazarit was significantly less than the vehicle control during the first 13 days of the dosing period, reaching a minimum at day 6 (50 mg/kg) or day 8 (100 mg/kg) before gradually increasing to levels comparable to the vehicle control by day 15. Food consumption was then maintained at levels comparable to the vehicle control for the remainder of the study. No significant effect on food consumption was observed with gemfibrozil. Treatment with sibutramine resulted in the expected decrease in food consumption. This decrease was initially more rapid than seen with romazarit, reaching a minimum within the first 2 days of the dosing period and gradually increasing to levels comparable to the vehicle control by day 8.

Increased liver weight was observed with romazarit at 50 and 100 mg/kg and, to a similar degree, with gemfibrozil. No increase in liver weight was seen with romazarit at 15 mg/kg or with sibutramine.

Collectively, these data indicate that weight loss due to romazarit at 50 or 100 mg/kg was associated with a decrease in food consumption, although this decrease was transient while the weight effect remained more stable even as food consumption returned to levels comparable with the vehicle control. Romazarit at 50 or 100 mg/kg provided greater weight loss than sibutramine at 10 mg/kg, whereas the fibrate gemfibrozil at 50 mg/kg had no effect on weight or food consumption in this study.

### Example 5

### Romazarit Improved Glucose Tolerance in Rats with Diet-Induced Obesity

As part of the study described in Example 4, an oral glucose tolerance test (OGTT) was performed on day 21 of the dosing period, after a 2-4 hour fast. Dextrose (2 g/kg) was administered by oral gavage. Blood for glucose analysis was collected at 0, 15, 30, 60, 120 and 180 minutes after dextrose administration. At each time point, 0.25 ml whole blood was collected in EDTA, processed to plasma, and analyzed for blood glucose with a low-volume glucose kit.

Data are presented in Table 1, which shows for each treatment the baseline (0 minutes) glucose concentration, the maximum glucose concentration and the time at which that maximum was observed. Table 1 also shows the calculated “glucose excursion,” i.e., the increase in glucose concentration from baseline to the time of maximum glucose concentration.

TABLE 1
Results of oral glucose tolerance test

| | Time to | Glucose concentration | | |
|---|---|---|---|---|
| | maximum | (mg/dl) | | |
| Treatment | (minutes) | baseline | maximum | excursion |
| vehicle control | 60 | 146.6 | 232.5 | 85.9 |
| romazarit 15 mg/kg | 15 | 144.6 | 230.1 | 85.5 |
| romazarit 50 mg/kg | 15 | 142.2 | 203.3 | 61.1 |
| romazarit 100 mg/kg | 60 | 142.4 | 185.1 | 42.7 |
| gemfibrozil 50 mg/kg | 30 | 153.9 | 264.9 | 111.0 |
| sibutramine 10 mg/kg | 30 | 126.9 | 201.3 | 74.4 |

Romazarit did not, at any of the tested doses, affect baseline glucose concentration, but exhibited a dose-dependent effect on glucose excursion. At the lowest romazarit dose (15 mg/kg) the glucose excursion was comparable to the vehicle control. At 50 and 100 mg/kg romazarit, the glucose excursion was reduced from 85.9 mg/dl (vehicle control) to 61.1 and 42.7 mg/dl, respectively. The effect of romazarit at these doses was greater than seen with sibutramine (74.4 mg/dl). Gemfibrozil treatment resulted in a slightly elevated baseline glucose concentration and an elevated glucose excursion in the OGTT (110.0 mg/dl).

Results of this study indicated that romazarit could improve response of rats with diet-induced obesity to glucose challenge via OGTT, an effect not seen with the fibrate drug gemfibrozil.

### Example 6

### Romazarit Lowered Plasma Lipid Levels in Rats with Diet-Induced Obesity

As part of the study described in Example 4, a terminal blood sample (0.5 ml whole blood in EDTA) was collected on day 24, after a 12-18 hour fast. Whole blood was processed to serum, and analyzed for glucose, total cholesterol, HDL cholesterol, free fatty acids and total triglycerides.

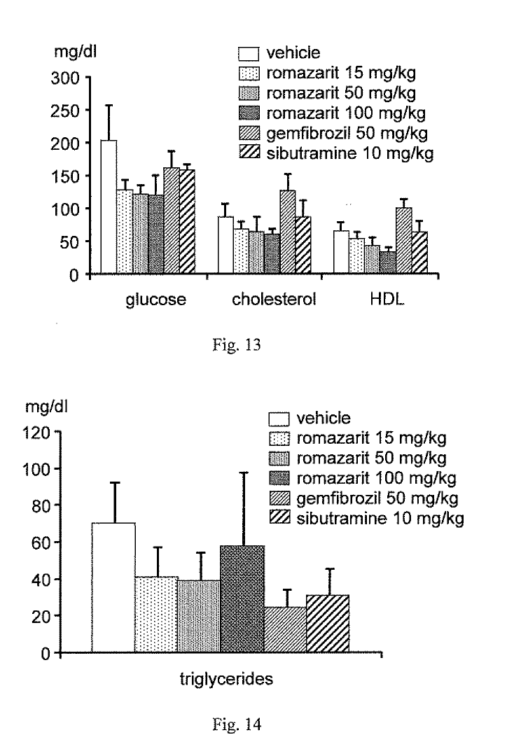

Terminal fasting serum glucose (FIG. 13) was significantly reduced in each of the three romazarit treatment groups compared to the vehicle control group. Gemfibrozil did not result in a significant decrease in fasting glucose. Sibutramine provided a significant decrease in fasting glucose but not of the same magnitude as romazarit.

Terminal cholesterol levels (FIG. 13) were reduced in a dose-dependent manner by romazarit compared to the vehicle control. The effect at the lowest romazarit dose (15 mg/kg) was not statistically significant. In contrast, cholesterol levels at 50 and 100 mg/kg romazarit were significantly reduced compared to the vehicle control. Treatment with sibutramine (10 mg/kg) had no effect on total cholesterol, while treatment with gemfibrozil (50 mg/kg) significantly increased total cholesterol.

Terminal HDL levels (FIG. 13) were reduced in a dose-dependent manner by romazarit compared to the vehicle control. The effect at the lowest romazarit dose (15 mg/kg) was not statistically significant, but HDL levels at 50 and 100 mg/kg romazarit were significantly reduced compared to the vehicle control. Treatment with sibutramine (10 mg/kg) had no significant effect on HDL, while treatment with gemfibrozil (50 mg/kg) significantly increased HAL.

Romazarit-induced reduction in HDL level is believed to be reflective of the overall reduction in plasma cholesterol. Fibrate administration in rodents has been shown to result in diminished hepatic production of apoliprotein A-I (apo A-I), the major component of HDL; however, this effect is counter to that on hepatic apo A-I secretion in humans, where fibrates cause an increase rather than decrease in apo A-I. See Berthou et al. (1996) J. Clin. Invest. 97:2408-2416.

Romazarit did not, at any of the tested doses, affect terminal levels of free fatty acids compared to the vehicle control (data not shown). Of the positive control treatments, sibutramine likewise had no effect, but gemfibrozil gave a significant increase in free fatty acid levels.

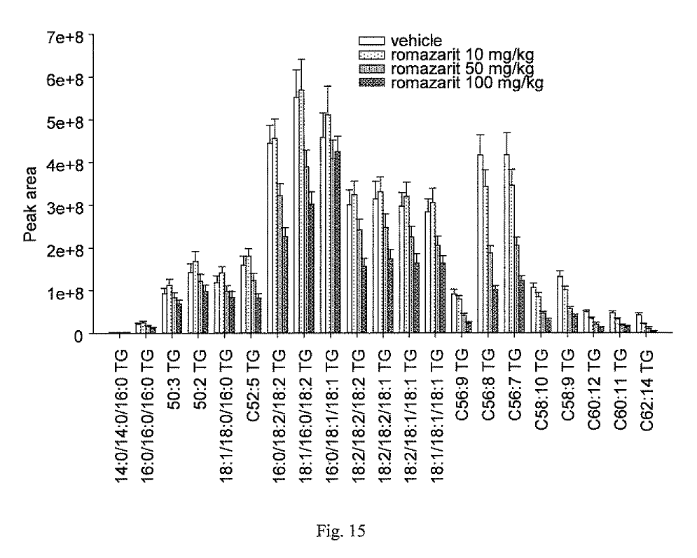

Treatment with romazarit at 15 and 50 mg/kg resulted in significant reduction in terminal fasting levels of triglycerides compared to the vehicle control, but the effect seen at the 100 mg/kg dose was apparently smaller and did not achieve statistical significance in this study (FIG. 14). Both sibutramine (10 mg/kg) and gemfibrozil (50 mg/kg) resulted in significant reduction of terminal fasting triglyceride levels.

Collectively, the data from this study indicate potential efficacy of romazarit for reducing blood levels of cholesterol and triglycerides.

### Example 7

### Romazarit did not Provide Weight Loss or Affect Glucose or Lipid Levels in a Diabetic (db/db) Mouse Model

A study was conducted to evaluate effects of romazarit (15, 50 and 100 mg/kg, once daily) on diabetic and metabolic syndrome parameters in a diabetic-prone (db/db) mouse model with a high-fat diet. The fibrate PPARα agonist fenofibrate (50 mg/kg) and the PPARγ agonist rosiglitazone (10 mg/kg) were used as reference controls for the study.

This model did not yield results confirming the data seen in rats (Examples 4-6 above). None of the romazarit or fenofibrate treatments reduced weight gain. As expected of a PPARγ agonist, rosiglitazone caused substantial increase in weight gain. Rosiglitazone reduced whole blood glucose levels, but no such effect was seen with romazarit at any of the doses tested or with fenofibrate. An OGTT to measure response to glucose challenge was inconclusive, in part because of very high baseline glucose levels. Fenofibrate tended to decrease total plasma triglycerides but increase total cholesterol and HDL; romazarit showed a tendency likewise to reduce total plasma triglycerides at higher doses and did not significantly affect total cholesterol or HDL.

### Example 8

### Romazarit Exhibited Effects on Plasma Lipids Similar to Fibrates in Normal Rats

### Objective of Study

The objective of this study was evaluation of modulation of plasma lipids by romazarit relative to the fibrate drugs bezafibrate, gemfibrozil and fenofibrate in normal (non-obese) rats.

### Study Methods

The study design was based on the protocols used in Examples 1-3. Male Sprague-Dawley rats were provided normal chow ad libitum, both before and during the study period. Rats received romazarit, bezafibrate, gemfibrozil, fenofibrate or vehicle alone via oral gavage (p.o.) once per day over a period of 7 days. Each compound was administered at 50 and 100 mg/kg; romazarit was additionally administered at the low dose of 10 mg/kg. Six rats were assigned to each treatment group (8 to the vehicle control). The study terminated on the day following administration of the seventh dose (day 8), with terminal blood collected for plasma lipid and lipoprotein analyses including LC-MS-based determination of lipid profiles, total and free cholesterol analyses, and lipoprotein cholesterol determinations (VLDL, LDL and HDL).

### Results

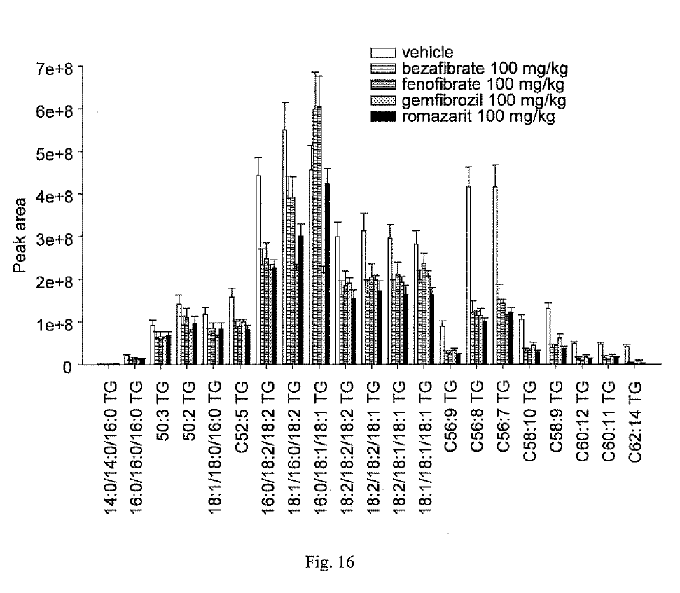

Romazarit significantly lowered plasma triglycerides (FIG. 15) consistent with previous data. The effect was substantially equipotent with that of the fibrate drugs bezafibrate, gemfibrozil and fenofibrate (FIG. 16). In FIGS. 15 and 16, different triglyceride moieties are identified by abbreviations derived as follows. Fatty acyl carbon content and unsaturation are denoted as x:y where x=number of carbon atoms and y=number of double bonds. Where these parameters have been identified for each of the three constituent fatty acids in the triglyceride, they are shown individually. Where data are available only for the total number of acyl carbons and double bonds in all three constituent fatty acids, only one x:y descriptor is shown, representing the total in each case.

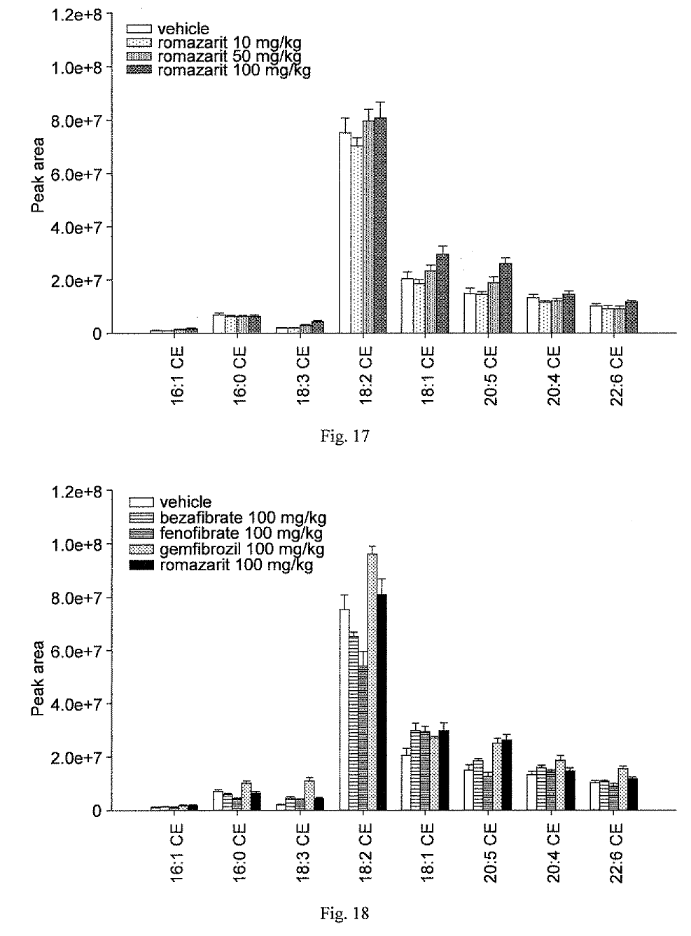

Also consistent with previous observations, romazarit increased at least some fractions of plasma cholesterol esters (FIG. 17), an effect that was common to all the compounds tested in this study, although to varying degrees (FIG. 18). In FIGS. 17 and 18, different cholesterol esters are identified by abbreviations derived as follows. Fatty acyl carbon content and unsaturation are denoted as x:y where x=number of carbon atoms and y=number of double bonds.

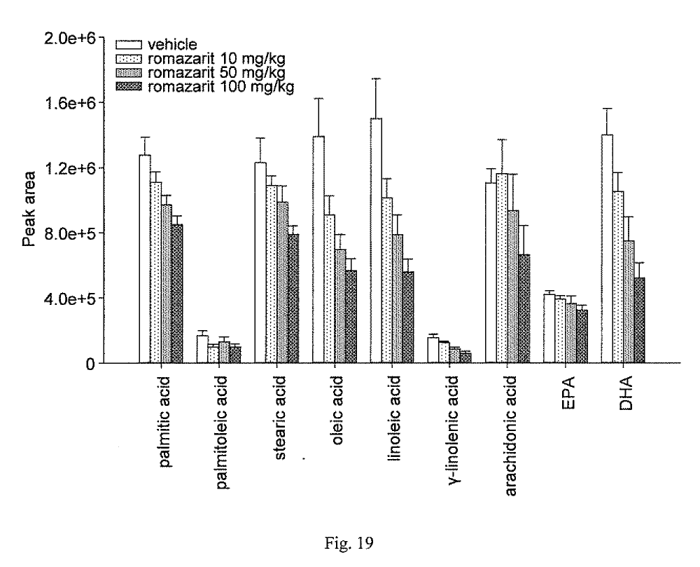

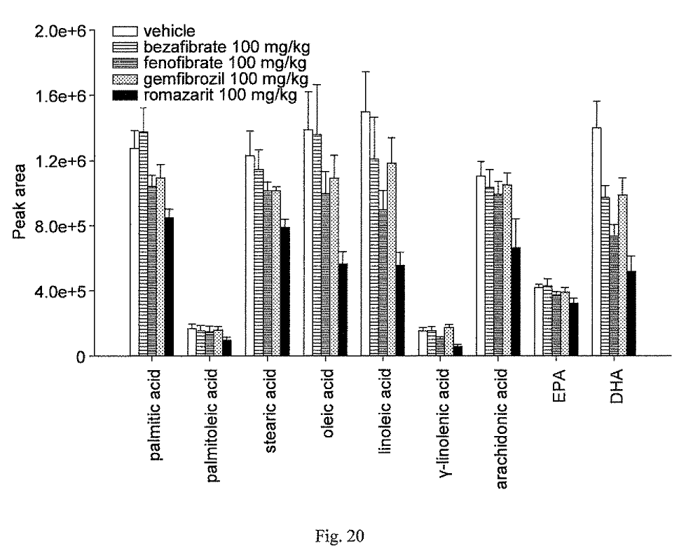

In addition, romazarit lowered plasma free fatty acids in a dose-dependent manner (FIG. 19) and displayed superior potency in this regard compared to the other compounds tested (FIG. 20).

In order to elucidate the impact of romazarit on plasma lipoproteins, an ultraviolet (UV) size exclusion chromatography (SEC) system was used to separate plasma into broad lipoprotein-containing fractions that were based on relative retention times of human VLDL (very low density lipoprotein), LDL (low density lipoprotein), and HDL (high density lipoprotein) standards. These fractions were collected for cholesterol assays and the UV chromatograms were integrated to provide relative quantitation of the three lipoprotein types. It is important to note that since the UV absorbance measurement is not specific for lipoproteins and may also derive from other plasma constituents that co-elute with a particular lipoprotein, the relative quantitation data reported here is an approximation. The HDL fraction in particular may have included such co-eluting species, as evidenced by the relatively high integrated area values for this fraction.

The results of these analyses indicated that romazarit significantly lowered VLDL, an effect consistent with the robust plasma triglyceride-lowering effect shown above. Of the other test compounds, only gemfibrozil displayed a similarly significant effect. With respect to HDL, none of the test compounds showed a significant effect as assayed using this SEC methodology. The lack of an observed effect on HDL is surprising in view of the generally appreciated ability of fibrates to increase HDL level in plasma; these results may reflect the aforementioned weakness of the quantitative methodology used here.

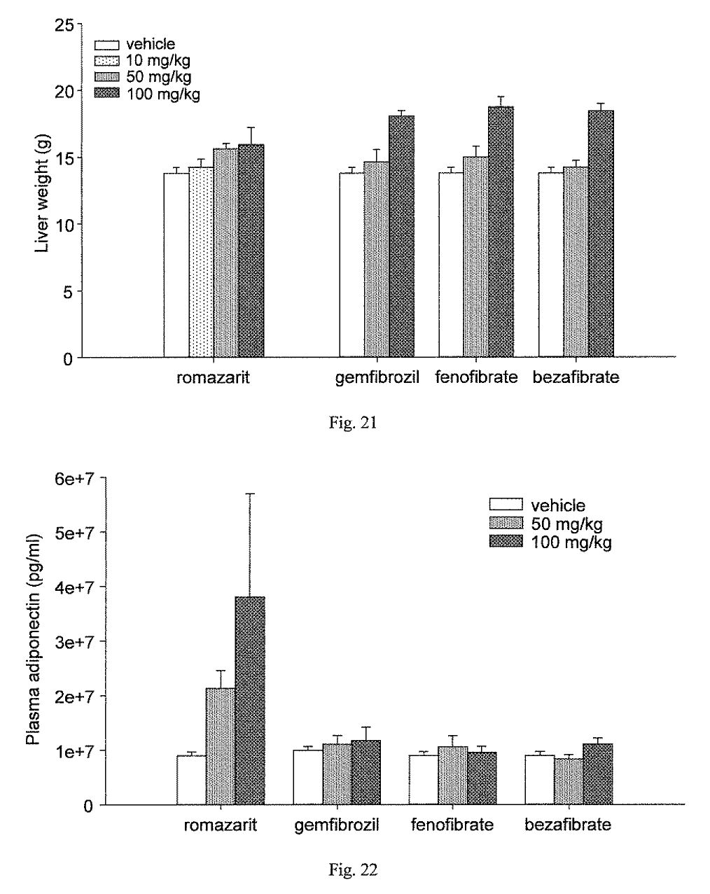

Animals were sacrificed and liver weights measured at conclusion of the study. Each of the test compounds gave rise to increased liver mass (FIG. 21), perhaps, at least in part, as a result of agonism of PPARα. At the highest dose (100 mg/kg), the effect of romazarit on liver weight was less pronounced than that of bezafibrate, gemfibrozil or fenofibrate.

### Example 9

### Romazarit Improved Indicators of Insulin Sensitivity in Normal Rats

As part of the study described in Example 8, analyses were also performed on various metabolic parameters. Results from these analyses indicated that romazarit had superior in vivo potency compared to the fibrate compounds and gave further support to a potentially unique application in metabolic disease. For example, romazarit dose-dependently increased plasma adiponectin (FIG. 22). Adiponectin is an insulin-sensitizing protein that is abundantly expressed in adipocytes and is secreted into the circulation (see review by Lara-Castro et al. (2007) cited above). PPARγ agonists of the thiazolidinedione class have been shown to significantly upregulate adiponectin, raising the question as to whether romazarit acts in a similar manner. However, molecular pharmacology studies have indicated that romazarit is not a PPARγ agonist (data not shown), suggesting it acts through a different mechanism.

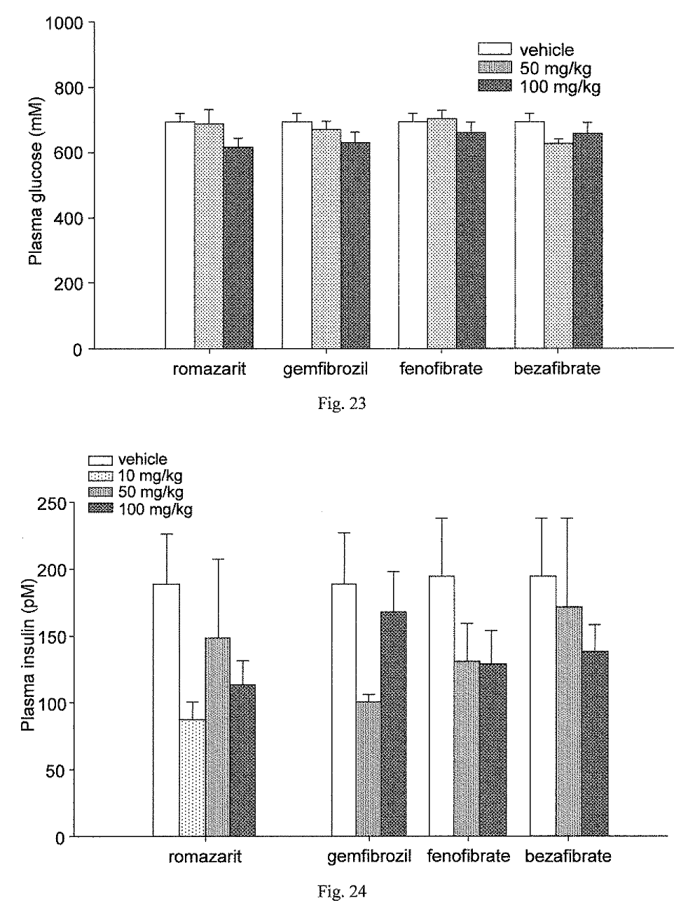

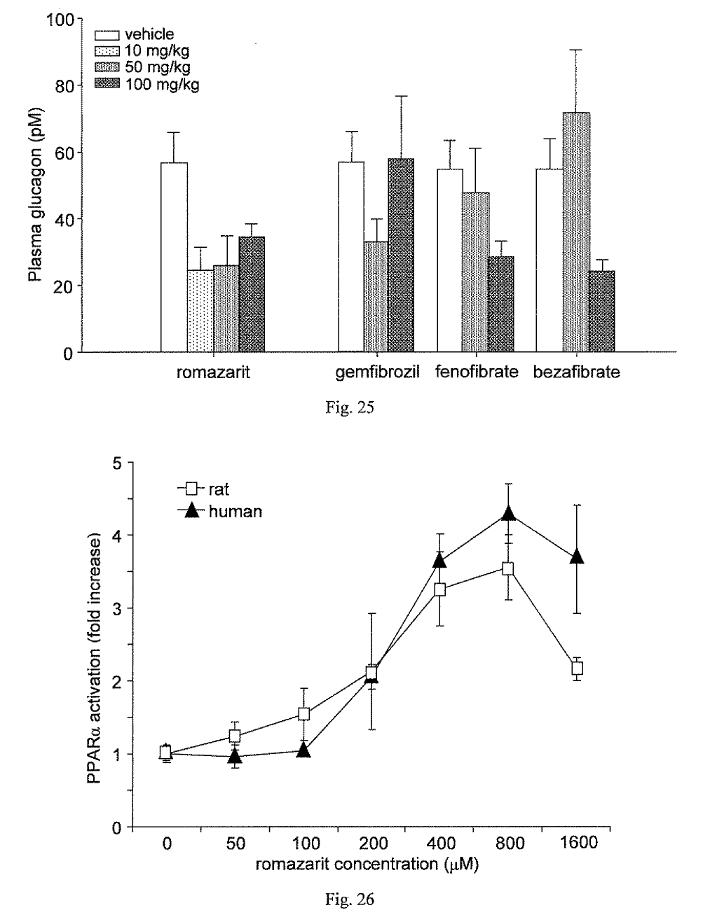

Further indication of potential for romazarit to improve insulin sensitivity was its effect to lower plasma free fatty acids (FIGS. 19 and 20, vide supra) as elevated levels of free fatty acids contribute to insulin resistance. Romazarit appeared to lower both glucose and insulin in plasma (FIGS. 23 and 24, respectively). Finally, romazarit also lowered plasma glucagon (FIG. 25). Glucagon typically rises in response to lowered plasma glucose and stimulates glycogen metabolism in the liver. Romazarit appears to alter this process.

### Example 10

### Romazarit Activated Rat and Human PPARα

The effects of romazarit shown above on plasma lipids and on weight gain are consistent with agonism of the nuclear hormone receptor PPARα, a known mechanism of action of the fibrate class of drugs. However, as indicated hereinabove, romazarit was originally developed not as an antidyslipidemic or anti-obesity drag but as an antirheumatic agent, and any effect of romazarit on PPARα has not, to the best of the present inventors' knowledge, hitherto been reported.

A study was therefore conducted to determine whether romazarit exhibits PPARα agonism properties. For this study, a luciferase reporter plasmid containing 5 copies of a defined genetic regulatory PPARα response element (PPARRE) was utilized to detect romazarit-induced PPARRE activation in presence of a co-activator, retinoic acid. Specifically, HepG2 cells were transfected with expression vectors for either human or rat PPARα and the PPARRE-luciferase reporter plasmid. After 24 hours, the cells were trypsanized and subsequently seeded in 96-well clear-bottom plates at a density of 1.0×10⁵ cells/ml. The cells were then treated with retinoic acid (200 nM) alone or with increasing concentrations of romazarit, in three replicates.

Romazarit was found to activate both human (hPPARα) and rat (rPPARα) isoforms with an EC₅₀ of approximately 200 μM (FIG. 26).

In a parallel study (not reported in detail herein), romazarit did not exhibit activity as an agonist of PPARγ.

All patents and publications cited herein are incorporated by reference into this application in their entirety.

The words “comprise”, “comprises”, and “comprising” are to be interpreted inclusively rather than exclusively.

What is claimed is:

1. A method for treating a condition of low metabolic rate, dyslipidemia, excess adiposity and/or insulin resistance, or a disease or syndrome characterized by one or more of said conditions in a subject, comprising administering to the subject a therapeutically effective amount of romazarit or a pharmaceutically acceptable salt, ester or prodrug thereof.

2. The method of claim 1, wherein the condition, disease or syndrome is accompanied by an inflammatory or pro-inflammatory condition.

3. The method of claim 1, wherein the condition, disease or syndrome comprises one or more of (a) elevated plasma triglycerides, (b) elevated total cholesterol, (c) depressed HDL cholesterol, (d) elevated plasma amino acids, (e) elevated plasma thyroxine and/or depressed plasma triiodothyronine, (f) elevated fasting glucose, (g) impaired glucose tolerance, (h) impaired insulin sensitivity, (i) excess visceral adiposity, and (j) overweight or obesity.

4. The method of claim 1, wherein the subject has dyslipidemia, obesity or a combination thereof.

5. The method of claim 1, wherein the subject is identified as having metabolic syndrome.

6. The method of claim 1, wherein the romazarit or salt, ester or prodrug thereof is administered in an amount effective to achieve at least one of (a) a reduction in plasma amino acids; (b) a reduction in plasma thyroxine and/or an increase in plasma triiodothyronine; (c) a reduction in plasma triglycerides; (d) an increase in plasma HDL or cholesterol esters indicative thereof; (e) a reduction in fasting glucose level in plasma;
(f) enhanced glucose tolerance; (g) an increase in plasma adiponectin; (h) reduction of excess visceral adiposity; and (i) inhibition of weight gain.

7. The method of claim 1, wherein the romazarit or salt, ester or prodrug thereof is administered in a daily romazarit dose of about 1 to about 100 mg/kg body weight of the subject.

8. The method of claim 1, wherein the subject is an adult human and the romazarit or salt, ester or prodrug thereof is administered in a daily romazarit dose of about 50 to about 5000 mg.

9. The method of claim 1, wherein the romazarit or salt, ester or prodrug thereof is administered via an intravenous, subcutaneous, intradermal, transdermal, rectal, intraoral, intranasal or peroral route.

10. The method of claim 9, wherein the route of administration is peroral.

11. The method of claim 1, wherein the romazarit or salt, ester or prodrug thereof is administered in combination therapy with at least one additional agent that is (a) effective for treatment of a component of the disease or syndrome, (b) effective for treatment of a condition secondary to the disease or syndrome, (c) effective for treatment of a condition to which the disease or syndrome is secondary, or (d) a causal agent, as an adverse side-effect, of the disease or syndrome.

12. The method of claim 11, wherein the at least one additional agent is selected from the group consisting of antidiabetic agents, antihypertensive agents, anti-obesity agents, antihyperlipidemic agents, and combinations thereof.

13. A method for reducing risk or future incidence or severity of at least one condition secondary to dyslipidemia and/or obesity in a subject having dyslipidemia, obesity or a combination thereof, comprising administering to the subject romazarit or a pharmaceutically acceptable salt, ester or prodrug thereof in an amount therapeutically effective for treatment of the dyslipidemia, obesity or combination thereof.

14. The method of claim 13, wherein the at least one secondary condition is selected from the group consisting of hypertension, ischemic heart disease, cardiomyopathy, cardiac infarction, stroke, venous thromboembolic disease, pulmonary hypertension, breast cancer, prostate cancer, bowel cancer, endometrial cancer, obesity-hypoventilation syndrome, asthma, obstructive sleep apnea, low back pain, osteoarthritis of weight-bearing joints, depression, sexual dysfunction, erectile dysfunction, infertility, obstetric complications, fetal abnormalities, gallstones, gout, non-alcoholic steatohepatitis, urinary incontinence, gastroesophageal reflux, venous ulcer, stasis ulcer, intracranial hypertension, accident proneness, skin disorders and combinations thereof.
